(19) United States
(12) Patent Application Publication
(10) Pub. No.: US 2008/0312721 A1
(43) Pub. Date: Dec. 18, 2008

(54) Tanning Booth

(76) Inventor: Jason Lemieux, Winnipeg (CA)

Correspondence Address: ADE & COMPANY INC., 2157 Henderson Highway, WINNIPEG, MB, R2G1P9, CA

(21) Appl. No.: 11/762,436

(22) Filed: Jun. 13, 2007

(51) Int. Cl. A61N 5/06

(52) U.S. Cl. 607/91; 607/94

(57) ABSTRACT

A tanning booth comprises a housing with upright walls having an inner reflective surface surrounding a hollow interior. A plurality of high strength tanning bulbs arranged to emit ultraviolet light are supported circumferentially about the hollow interior adjacent a top end of the upright walls and oriented to direct the ultraviolet light downwardly and inwardly into the hollow interior of the housing. This configuration of bulbs with reflective walls permits even distribution of ultraviolet light for even tanning, for example even when the user is performing exercise on an exercise machine within the hollow interior. A source of concentrated oxygen can be arranged to be breathable by a user located in the hollow interior of the housing for additional benefits to the user.

This application claims the benefit under 35 U.S.C. 119(e) of U.S. provisional application Ser. No. 60/812,993, filed Jun. 13, 2006.

### FIELD OF THE INVENTION

The present invention relates to a tanning booth in which tanning bulbs are supported above a hollow interior of the booth for receiving an occupant therein in which the hollow interior is surrounded by upright reflective walls.

### BACKGROUND

Tanning beds are well known in which users are exposed to concentrated ultraviolet light to promote tanning of the users' skin. Common designs of tanning booths include horizontal beds including a lower panel upon which the user lies and an upper panel suspended above the user. Each panel comprises a plurality of ultraviolet bulbs for simultaneously tanning both front and back sides of a user lying on the bed. In a further configuration of a tanning booth, upright walls surround a user in a standing position in close proximity to the user and tanning is provided by bulbs positioned along a full height of the wall to produce an even tan. These known designs are generally very small and constrictive to the user as the user must be positioned very close to the filter glass which covers the bulbs. Another known disadvantage is that a plurality of expensive bulbs must be positioned in close proximity to one another in order to achieve an even distribution of ultraviolet tanning light according to conventional tanning booth designs.

U.S. Pat. No. 5,957,959 to Rissmaney et al discloses a home tanning apparatus in which a flexible wall panel is provided which locates a plurality of closely spaced bulbs extending the full length of the wall to function similarly to known stand up tanning booths. When standing within the apparatus, no ultraviolet light is provided from above the user.

U.S. Pat. No. 4,582,062 to Albini discloses a home tanning tent structure in which an elongate tent structure is provided for closely fitting a single occupant laying therein. A single bulb is mounted centrally above the user for cooperation with reflective walls sloping downwardly and outwardly away from the bulb. The use of a single bulb does not evenly distribute ultraviolet light to the user and it requires the user to be positioned within a small constrictive space in close proximity to the reflective walls in order to obtain any benefit from the tanning structure.

U.S. Pat. No. 6,478,810 to Purschel discloses a tanning booth in which a reflective dome is positioned above a user in a recumbent position. A plurality of ultraviolet lights must be positioned at the base of the dome to be reflected upwardly into the dome and back onto the user so that the user does not receive any direct light from the bulbs. Relying only on reflected light reduces the strength of the ultraviolet light exposure to the user and accordingly increases the required exposure time to effectively obtain a tan. In order to be useful, the reflector must be positioned in close proximity to the user so that the tanning booth is very constrictive to the user.

In general, known designs of tanning booths require direct light from the bulbs with a close spacing of the bulbs relative to the user so that a resulting constrictive space results and the user is effectively prevented from making use of the tanning time to accomplish any other activities simultaneously.

### SUMMARY OF THE INVENTION

According to one aspect of the invention there is provided a tanning booth comprising:

a housing comprising upright walls having an inner reflective surface surrounding a hollow interior of the housing;

a plurality of high strength tanning bulbs arranged to emit ultraviolet light, the bulbs being supported circumferentially about the hollow interior adjacent a top end of the upright walls and being oriented to direct the ultraviolet light downwardly and inwardly into the hollow interior of the housing.

According to a second aspect of the present invention there is provided a tanning booth comprising:

a housing comprising upright walls surrounding a hollow interior;

a plurality of high strength tanning bulbs arranged to emit ultraviolet light, the bulbs being supported circumferentially about the hollow interior and being oriented to direct the ultraviolet light into the hollow interior of the housing; and

an exercise machine supported within the hollow interior of the housing upon which a user can perform cardiovascular exercise.

According to a third aspect of the present invention there is provided a tanning booth comprising:

a housing comprising upright walls surrounding a hollow interior;

a plurality of high strength tanning bulbs arranged to emit ultraviolet light, the bulbs being supported circumferentially about the hollow interior and being oriented to direct the ultraviolet light into the hollow interior of the housing; and

a source of concentrated oxygen arranged to be breathable by a user located in the hollow interior of the housing.

By providing a tanning structure having upright walls with inner reflective surfaces and which support circumferentially spaced tanning bulbs thereabove, the tanning booth is more accommodating for providing more space to the user to perform other activities within the tanning booth while tanning while also providing an even distribution of ultraviolet light to the user. When the bulbs are mounted only in the roof in particular, much higher strength bulbs can be used which are plural times stronger than conventionally used tanning bulbs to safely ensure a sufficient separating distance between the user and the bulbs that the user within the booth may perform exercise for example. In some embodiments, the tanning booth may be provided with an exercise machine such as a recumbent bicycle so that the user may save time by accomplishing two tasks at once. The tanning booth may be further improved when provided with a source of concentrated oxygen which is supplied either directly to the user or to the surrounding environment in the hollow interior of the booth to obtain the many benefits of breathing concentrated oxygen at the same time as tanning. Breathing concentrated oxygen while tanning has beneficial effects for improving the quality of the tan obtained from the ultraviolet light.

A target tanning area may be defined within the hollow interior which is spaced several inches below the tanning bulbs and across which ultraviolet light distribution is substantially constant in which the target tanning area is spaced inwardly from the upright walls by several inches.

There may be provided the barrier surrounding the target area.

The inner reflective surface of the upright walls is preferably substantially uninterrupted and is generally cylindrical about the hollow interior.

The bulbs are preferably only supported above the hollow interior.

There may be provided a filter member for filtering undesirable portions of the light spectrum emitted by the bulbs which is supported spaced below the bulbs by approximately 3 to 9 inches.

The bulbs may be supported within a common ventilated area above the hollow interior.

Preferably each bulb is partially surrounded by a reflector which directs light substantially only downward and inward into the hollow interior.

Each reflector may include a top wall above the respective bulb and an exterior wall extending downwardly from the top wall adjacent an exterior side nearest to the upright walls.

The exterior wall of the reflector may extend downwardly at an inward incline away from the walls of the housing.

Each reflector may also include an inner wall extending downwardly at an inward incline at an inner side of the bulb.

Each reflector is preferably open at respective ends to permit circulation of cooling air therethrough.

The tanning bulbs preferably comprise high pressure metal halide bulbs.

The housing may include a floor supporting the upright walls to extend upwardly from a periphery thereof which is elongate in one direction and has a generally oval shape.

Preferably there is provided a door mounted within a door opening in the upright walls in which the door has a reflective inner surface.

The housing preferably also includes an inner reflective surface on the floor.

All of the bulbs are preferably supported near 7 feet or greater in height above the floor of the housing.

The booth may be provided in combination with an exerciser supported within the hollow interior in which the exerciser comprises a recumbent stationary bicycle including a seat having a transparent back portion and a transparent seating surface.

There may be provided a source of concentrated oxygen arranged to be breathable by a user within the hollow interior.

The oxygen source may include tubes for communication directly with a mouth or nose of the user, or alternatively, the hollow interior of the housing may be substantially fully enclosed and the oxygen source communicates with the hollow interior.

One embodiment of the invention will now be described in conjunction with the accompanying drawings in which:

### BRIEF DESCRIPTION OF THE DRAWINGS

FIG. 1 is a side elevational view of the tanning booth.

FIG. 2 is a bottom plan view of the tanning booth.

FIG. 3 is an end elevational view of the tanning booth.

FIG. 4 is a bottom plan view of the lighting hood supported in the roof of the tanning booth.

FIG. 5 is a partly sectional elevational view of one of the tanning bulbs along an interior side thereof shown with an associated reflector housing being removed.

FIG. 6 is an end elevational view of one of the tanning bulbs shown with the reflector housing mounted thereon.

FIG. 7 is a bottom plan view of the reflector housing.

FIG. 8 is an outer side elevational view of the reflector housing.

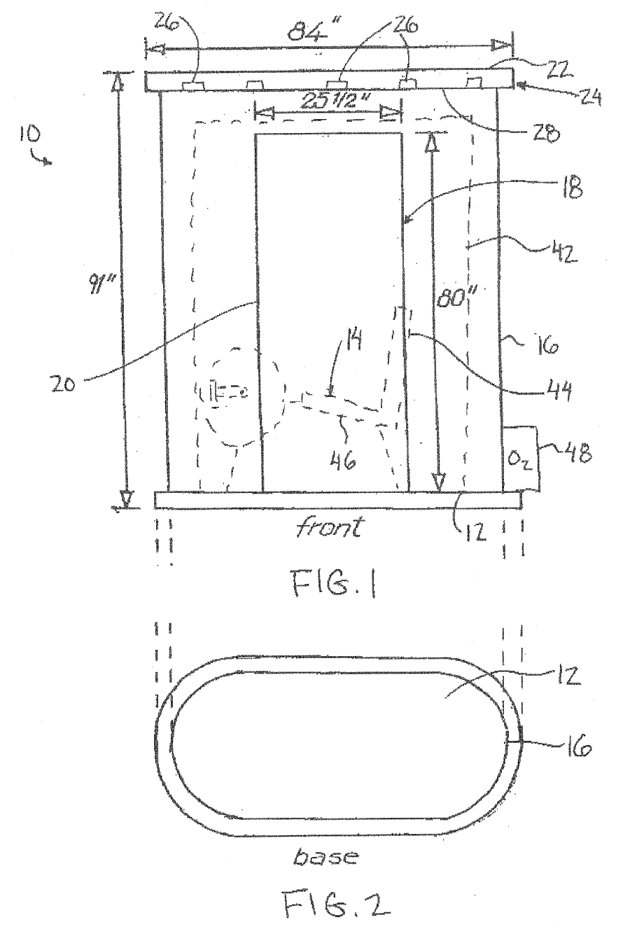

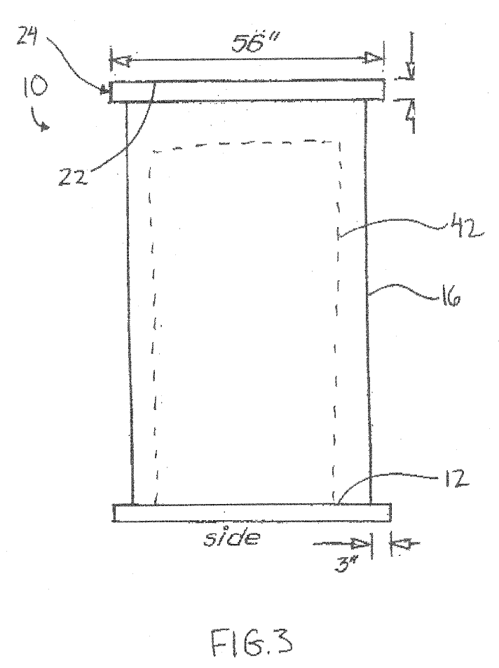

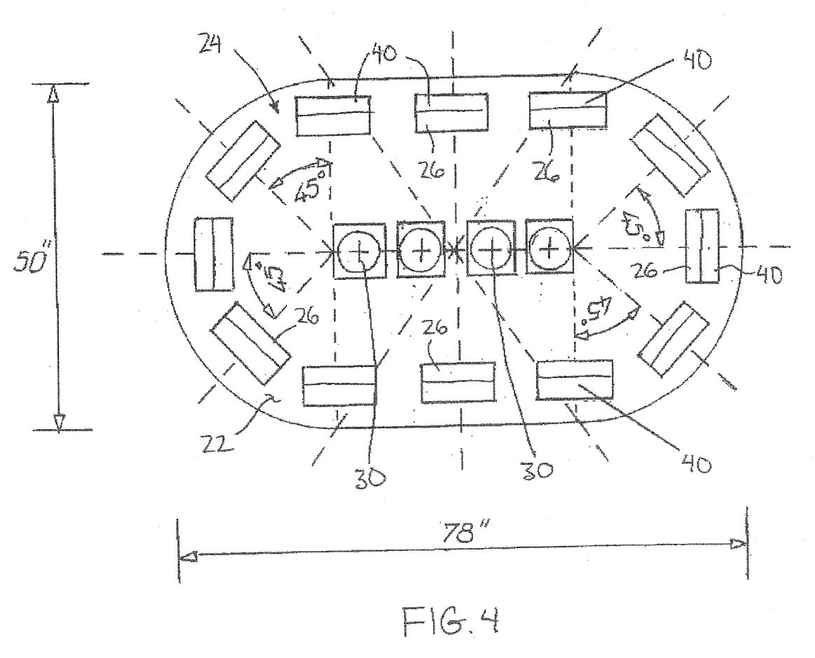

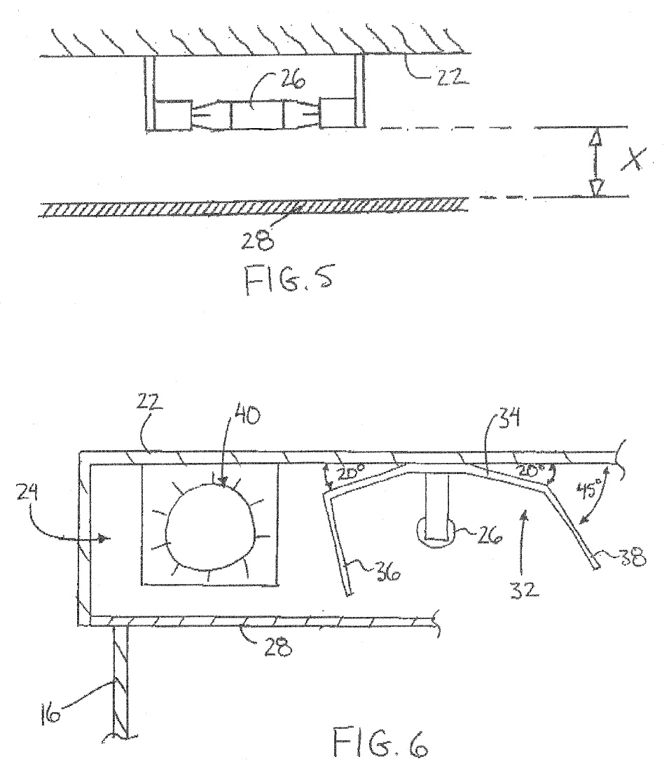

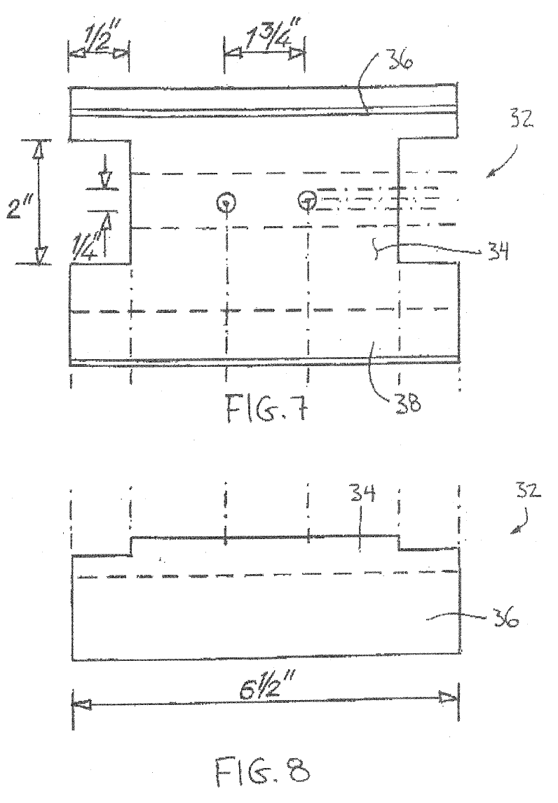

In the drawings like characters of reference indicate corresponding parts in the different figures.

### DETAILED DESCRIPTION

Referring to the accompanying figures there is illustrated a tanning booth generally indicated by reference numeral 10. The booth 10 is particularly suited for providing an even tan to a user situated within a large enclosure so that the user can simultaneously perform exercise or other activities within the comfort of the booth and without the constrictive space normally provided in a tanning booth. Larger space is achieved by providing higher strength tanning bulbs than are used conventionally and which are positioned at greater spacing than conventional tanning beds.

The tanning booth includes a housing having a floor 12 which is supported horizontally and which is suitably reinforced for supporting a person or exercise equipment 14 thereon. The top surface of the floor 12 at an interior side is reflective. The floor is elongated in one direction with rounded ends so that the floor has a generally oval shape.

Upright walls 16 are provided about a full periphery of the floor to extend upwardly therefrom in a vertical orientation joined with the floor 12 forming the base of the housing. The upright walls 16 have a substantially uninterrupted generally cylindrical inner surface which is reflective and which surrounds a hollow interior 18 of the tanning booth. A side door 20 is hinged within a door opening at one side of the housing for access of persons into the hollow interior 18. The side door 20 includes a reflective lining which is continuous with the upright walls 16 when closed.

A roof 22 spans the top end of the upright walls 16 to fully enclose the hollow interior of the housing. In the illustrated embodiment the base has dimensions which are approximately 78 inches by 50 inches within the hollow interior of the housing. The overall outer dimension of the base and roof is approximately 84 inches×56 inches. Overall height of the housing is approximately 91 inches with the hollow interior providing approximately 6½ to 7 feet of clearance for a user to stand within the booth while maintaining an adequate spacing between a person of average height and a source of ultraviolet light in the roof. The door is approximately 80 inches tall by 25½ inches wide.

A lighting hood 24 is provided for mounting within the roof 22 for illuminating the hollow interior of the booth with ultraviolet light. The lighting hood includes a plurality of tanning bulbs 26 supported therein comprising conventional ultraviolet emitting bulbs used in the tanning industry but which have a considerably higher strength than bulbs typically used in tanning beds. The bulbs 26 comprise a plurality of metal halide bulbs which are circumferentially spaced about the periphery of the wall of the housing adjacent the walls 16 but spaced above the hollow interior which is surrounded by the walls. The bulbs are only mounted in the roof above the hollow interior so that all ultraviolet light originates from above and is directed downwardly and inwardly to the hollow interior with reflected ultraviolet light off of the inner surface of the upright walls 16 providing an even distribution of the light to the user.

The lighting hood 24 includes a common filter panel 28 which spans horizontally the full interior dimensions of the hollow interior with all of the bulbs 26 being supported thereabove for filtering of any undesirable portion of the light spectrum. The filter panel 28 defines a common area within the lighting hood 24 which supports all of the bulbs 26 therein. The filter panel 28 is supported approximately 6½ to 7 feet above the floor so that the filter panel remains spaced above the user in the booth by at least several inches for the average user.

A plurality of ventilation fans 30 are mounted centrally within the roof 22 above the lighting hood 24 for communication with the common area locating the bulbs therein and for ventilating heat from the common area to the exterior of the housing. The fans 30 are supported centrally in the lighting hood and may also be provided in communication with the hollow interior of the housing for ventilating heat to the exterior of the housing.

The bulbs are provided as a set of 12 which are supported at spaced positions along the elongate sides of the housing in which the bulbs along the sides are aimed downwardly and inwardly to the opposing side of the housing. At each end of the housing, bulbs are circumferentially spaced at approximately 45° intervals and are aimed downwardly and inwardly towards a centre of a radius of curvature defining the rounded periphery at the ends of the housing. In each instance, the bulbs 26 are oriented to direct the ultraviolet light emitted therefrom substantially only downwardly and inwardly towards the center of the hollow interior of the housing.

Each bulb includes a respective reflector housing 32 which acts to substantially direct light only downwardly and inwardly towards the center of the hollow interior of the housing. Each bulb 26 is positioned within a respective housing to extend substantially horizontally and perpendicularly to a portion of the wall 16 to which the bulb is nearest. The reflector housing comprises an elongate trough which extends parallel to the bulb to partially surround the top and elongate sides of the bulb. Each reflector housing has a top wall 34 comprised of three segments oriented at an obtuse angle relative to one another so that the top wall approximately forms a slight arc in profile but which is substantially horizontal as the segments at the outer sides are inclined downwardly from horizontal by only approximately 20°. An exterior wall 36 extends mostly downwardly from an exterior edge of the top wall 34 nearest to the wall 16 of the housing. The exterior wall 36 of the reflector housing is inclined inwardly towards the bottom free end thereof at an offset angle of approximately 10° from vertical.

An interior wall 38 is provided on each reflector housing 32 which extends from the interior edge of the top wall which is nearest to a center of the booth. The interior wall 38 extends downwardly and inwardly at an incline of approximately 45° from horizontal. The walls of the reflector housings are formed of reflective material open at opposing ends of the reflector housing to provide proper ventilation.

A fan 40 is associated with each bulb to be positioned directly adjacent the respective reflector housing 32, outward in relation to the exterior wall 36 of the reflector housing so as to be mounted between the respective bulb 26 and the cylindrical wall 16 of the tanning booth.

Once the bulbs are illuminated, the path of light projected by the reflector housings 32 associated with each bulb define a target tanning area surrounded by a boundary 42 which is spaced inwardly from the walls 16 of the booth and which is spaced below the filter panel in the roof by several inches, for example in the range 3 to 12 inches in each direction. Within this target tanning area defined by the boundary 42, the distribution of ultraviolet light is substantially uniform across a full height of the booth despite the lights only being mounted adjacent the roof due to the increased strength of the bulbs, the configuration of the reflector housings and the reflective properties of the surrounding walls 16 which are substantially uninterrupted. The boundary 42 may be indicated by suitable indicia on the floor and walls of the housing, or alternatively a barrier member may be provided which inhibits users from exiting the targeting tanning area in the form of a clear panel or other barrier which does not block ultraviolet light.

In the illustrated embodiment an exerciser 14 is provided in the form of a recumbent stationary bike upon which a user is seated to perform exercise. The seat includes a clear or transparent back 44 which permits ultraviolet light to penetrate therethrough to the user. Optionally a clear or transparent seating surface 46 may also be provided to similarly transmit ultraviolet therethrough.

To further enhance tanning, an oxygen source 48 is provided in the tanning booth to supply concentrated oxygen to the user either by tubes directly to the user's mouth or nose or by simply enriching the atmosphere within the booth with oxygen.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A tanning booth comprising:
a housing comprising upright walls having an inner reflective surface surrounding a hollow interior of the housing;
a plurality of high strength tanning bulbs arranged to emit ultraviolet light, the bulbs being supported circumferentially about the hollow interior adjacent a top end of the upright walls and being oriented to direct the ultraviolet light downwardly and inwardly into the hollow interior of the housing.

2. The booth according to claim 1 wherein a target tanning area is defined within the hollow interior which is spaced several inches below the tanning bulbs.

3. The booth according to claim 1 wherein there is provided a target tanning area across which ultraviolet light distribution is substantially constant in which the target tanning area is spaced inwardly from the upright walls by several inches.

4. The booth according to claim 1 wherein the inner reflective surface of the upright walls is substantially uninterrupted and is generally cylindrical about the hollow interior.

5. The booth according to claim 1 wherein the bulbs are only supported above the hollow interior.

6. The booth according to claim 1 wherein there is provided a filter member for filtering undesirable portions of the light spectrum emitted by the bulbs, the filter member being supported spaced below the bulbs by approximately 3 to 9 inches.

7. The booth according to claim 1 wherein the bulbs are supported within a common ventilated area above the hollow interior.

8. The booth according to claim 1 wherein each bulb is partially surrounded by a reflector which directs light substantially only downward and inward into the hollow interior.

9. The booth according to claim 8 wherein each reflector includes a top wall above the respective bulb and an exterior wall extending downwardly from the top wall adjacent an exterior side nearest to the upright walls.

10. The booth according to claim 9 wherein the exterior wall of the reflector extends downwardly at an inward incline away from the walls of the housing.

11. The booth according to claim 9 wherein each reflector includes an inner wall extending downwardly at an inward incline at an inner side of the bulb.

12. The booth according to claim 9 wherein each reflector is open at respective ends to permit circulation of cooling air therethrough.

13. The booth according to claim 1 wherein the tanning bulbs comprise high pressure metal halide bulbs.

14. The booth according to claim 1 wherein the housing includes a floor having an inner reflective surface.

15. The booth according to claim 1 wherein all of the bulbs are supported near 7 feet or greater in height above the floor of the housing.

16. The booth according to claim 1 in combination with an exerciser supported within the hollow interior.

17. The booth according to claim 16 wherein the exerciser comprises a seat having a seating surface and a back portion, at least one of the seating surface and the back portion being substantially transparent.

18. The booth according to claim 1 wherein there is provided a source of concentrated oxygen arranged to be breathable by a user within the hollow interior.

19. A tanning booth comprising:
a housing comprising upright walls surrounding a hollow interior;
a plurality of high strength tanning bulbs arranged to emit ultraviolet light, the bulbs being supported circumferentially about the hollow interior and being oriented to direct the ultraviolet light into the hollow interior of the housing; and
an exercise machine supported within the hollow interior of the housing upon which a user can perform exercise.

20. A tanning booth comprising:
a housing comprising upright walls surrounding a hollow interior;
a plurality of high strength tanning bulbs arranged to emit ultraviolet light, the bulbs being supported circumferentially about the hollow interior and being oriented to direct the ultraviolet light into the hollow interior of the housing; and
a source of concentrated oxygen arranged to be breathable by a user located in the hollow interior of the housing.
